(19) United States
(12) Patent Application Publication
(10) Pub. No.: US 2026/0269038 A1
(43) Pub. Date: Sep. 10, 2026

(54) GENERATIVE AI SYSTEM FOR ENHANCED RADIOLOGY REPORTS WITH COLORIZED MRI AND ILLUSTRATIVE OVERLAYS

(71) Applicant: EXPERT RADIOLOGY, New York, NY (US)

(72) Inventors: Avery J. Knapp, JR., New York, NY (US); Cristian Lorenzo, New York, NY (US)

(73) Assignee: EXPERT RADIOLOGY, New York, NY (US)

(21) Appl. No.: 19/660,363

(22) Filed: Apr. 28, 2026

Related U.S. Application Data
(63) Continuation of application No. PCT/US2025/045487, filed on Sep. 9, 2025.
(60) Provisional application No. 63/695,685, filed on Sep. 17, 2024.

(51) Int. Cl. G16H 15/00; G06T 7/00; G06T 11/10; G06T 11/60; G06V 20/50

(52) U.S. Cl. CPC G16H 15/00; G06T 7/0012; G06T 11/10; G06T 11/60; G06V 20/50; G06T 2207/10088; G06T 2207/20212; G06T 2207/30012; G06T 2210/41; G06V 2201/033

(57) ABSTRACT

Embodiments disclosed herein include software processes executed by a computer for ingesting and analyzing MRI image data and generating MRI-related reports. The report generation software executed by a computer generates MRI reports using medical data and MRI imagery data. The computer creates a text summary. Additionally, the computer generates the MRI report to include medical illustrations that overlay and merge with the MRI imagery data to visually explain the MRI results. Generative machine-learning models integrate colorized MRI images with the illustrations. The computer selects key MRI slices, colorizes them, matches them with relevant illustrations, and recursively refines the combined images to improve clarity and informational content. This process enhances the interpretability of radiology reports for both medical professionals and patients.

### CROSS REFERENCE TO RELATED APPLICATION

This application is a bypass continuation of PCT Application No. PCT/US2025/045487, filed Sep. 9, 2025, which claims the benefit of and priority to U.S. Provisional Application No. 63/695,685, filed Sep. 17, 2024, each of which is incorporated by reference in its entirety.

### TECHNICAL FIELD

This application generally relates to medical imaging and radiology, specifically to a system and method for improving radiology reports by adding key images, colorized images, illustration of images based upon the language in the radiologists' reports, as well as combining colorized magnetic resonance imaging (MRI) images with illustrative overlays using generative artificial intelligence (AI) and machine-learning architecture operations.

### BACKGROUND

Radiology reports are critical for diagnosing and treating various medical conditions. Traditional reports, however, can be challenging for non-specialists to interpret. This invention addresses this problem by integrating colorized MRI images and illustrative overlays to create more intuitive and informative reports.

### SUMMARY

Disclosed herein are systems and methods capable of addressing the technological shortcomings and may also provide any number of additional or alternative benefits and advantages. Embodiments include systems and methods for ingesting and analyzing MRI image data and generating MRI-related reports. The embodiments implement machine-learning architecture trained to generate the MRI reports. Beneficially, the report generation software executed by a computing device generates MRI reports using medical data and MRI imagery data ingested from medical imaging devices and other inputted data from medical provider devices or medical resource databases. The computer includes the machine-learning architecture trained to generate the MRI reports based on the various types of inputs and using terms or phrasing that are easier to understand for laypeople without a medical background. Additionally, the computer generates the MRI report to include illustrations that visually explain the findings of the MRI results, making the complex details of the MRI report much more accessible and easier to understand for a layperson to review. A generative AI may enhance MRI images and integrate the MRI images with dynamically selected, generated, or otherwise curated medical illustrations. The operations include selecting key MRI slices of the MRI imagery, colorizing the MRI slices, matching the MRI images with appropriate illustrations, and iteratively refining the combined images to improve clarity and informational content.

Embodiments may include computing system(s) and computer-implemented method(s) for enhancing radiology reports having medical imaging data, such as MRI imagery data. Embodiments may include an illustration database and a computer having at least one processor. The illustration database includes non-transitory machine-readable storage medium configured to store a plurality of illustrations images in a plurality of medical illustration image files having corresponding attributes related to a portion of human anatomy or a medical condition indicator. The computer having at least one processor may execute operations of obtain MRI imagery data containing one or more MRI images generated from an MRI imaging device. The computer may identify in the illustration database, a set of one or more medical illustration images based upon a set of one or more MRI slices of the one or more MRI images. The set of one or more medical illustration images being identified according to text of an input source report having the MRI imagery data. For each medical illustration image, the computer identifies a medical illustration image based upon at least one of an anatomical descriptor or a condition indicator in the input source report and associated with the medical illustration image. For each MRI image, the computer may generate an output image by combining an MRI image with the medical illustration image as identified in the illustration database using the one or more MRI slices of the MRI image. The computer may generate an output report having one or more output images.

The computer may generate a report summary based upon the text of the input source report. The computer generates the output report having the one or more output images and report summary.

When selecting the one or more MRI slices, for each MRI slice of the one or more MRI slices, the computer may generate a colorized instance of the MRI slice.

When generating the colorized instance, the computer may identify in the MRI slice a portion of an anatomy having a medical condition according to the condition indicator of the input source report. The computer may update a coloring of the portion of the anatomy having the medical condition.

When generating the colorized instance, the computer may receive a user input indicating a coloring a portion of an anatomy in the MRI slice having a medical condition.

When generating the output image, the computer may update the MRI imagery data containing the one or more MRI images based upon merging the MRI image and an overlay of the medical illustration image.

The computer may generate a refined image according to a graph representation of one or more image features, including an anatomical accuracy value and an illustration clarity value for the medical illustration image.

When identifying the set of one or more medical illustration images, the computer may select from the illustration database at least one of an axial image or a sagittal image based upon herniation data of a condition indicator.

The computer may receive the input source report having the MRI imagery data in a healthcare message data structure having a standard format. The computer may extract the input source report and the MRI imagery data from the healthcare message data structure.

The computer may select the one or more MRI slices from the MRI imagery data according to one or more input from a user device. Each MRI slice may include an overlay corresponding to one or more condition indicators of the input source report.

It is to be understood that both the foregoing general description and the following detailed description are exemplary and explanatory and are intended to provide further explanation of the invention as claimed.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1 shows components of a system for generating MRI reports using machine-learning architectures, according to an example embodiment.

FIG. 2 shows operations of a process for receiving medical image data and medical records data for machine-generated output reports using machine-learning architectures of a medical reporting system, according to an example embodiment.

FIGS. 3A-3C show operations of a process for executing machine-learning architectures of a medical reporting system trained for generating machine-generated output reports, according to an example embodiment.

FIG. 4 shows operations of a process for outputting medical image data and medical records data for machine-generated output reports using machine-learning architectures of a medical reporting system, according to an example embodiment.

FIG. 5 shows operations of a process for a computer-implemented method for enhancing radiology reports having medical imaging data, according to an example embodiment.

FIGS. 6-11 depict graphical user interfaces displaying portions of a source report and an output enhanced report generated by one or more machine-learning models of a machine-learning architecture, according to various example embodiments.

FIG. 6 depicts a sagittal view of a portion of the anatomy (e.g., T2 L3, L4), according to an example embodiment.

FIG. 7 depicts a sagittal view of a portion of the anatomy (e.g., T2 L4, L5), according to an example embodiment.

FIG. 8 depicts a sagittal view of a portion of the anatomy (e.g., T2 L5, S1), according to an example embodiment.

FIGS. 9A-9B depict the graphical user interfaces having machine-generated overlays for an enhanced version of the MRI images, according to example embodiments.

FIG. 10 depicts the graphical user interface displaying a machine-generated medical illustration image for a sagittal view (side view) of the spine, according to an example embodiment.

FIG. 11 depicts the graphical user interface displaying a machine-generated medical illustration image for an axial view (top-down view) of the spine at level L4-L5, according to an example embodiment.

### DETAILED DESCRIPTION

Reference will now be made to the example embodiments depicted in the drawings, and specific language will be used here to describe the same. It will nevertheless be understood that no limitation of the scope of the invention is thereby intended. Alterations and further modifications of the inventive features depicted and described here, and additional applications of the principles of the inventions as depicted and described here, which would occur to a person skilled in the relevant art and having possession of this disclosure, are to be considered within the scope of the invention.

Embodiments include systems and methods for ingesting and analyzing magnetic resonance imaging (MRI) image data and generating MRI-related reports. The embodiments implement machine-learning architecture trained to generate the MRI reports. Beneficially, the report generation software executed by a computing device generates MRI reports using medical data and MRI imagery data ingested from medical imaging devices and other inputted data from medical provider devices or medical resource databases. The computer includes the machine-learning architecture trained to generate the MRI reports based on the various types of inputs and using terms or phrasing that are easier to understand for laypeople without a medical background. The computer may take text from an MRI report, which is often complex and full of medical jargon, and create a summary that highlights the important information in a simple and concise manner. Additionally, the computer generates the MRI report to include medical illustration images that visually explain the findings of the MRI results, making the complex details of the MRI report much more accessible and easier to understand for a layperson to review.

In some embodiments, a computer executes machine-learning architecture for enhancing radiology reports using generative AI machine-learning models of the machine-learning architecture trained and programmed to integrate colorized MRI images with curated medical illustrations. The computer selects key MRI slices, colorizes them, matches them with relevant illustrations, and recursively refines the combined images to improve clarity and informational content. This process enhances the interpretability of radiology reports for both medical professionals and patients. The computer may be hosted in a reporting system, which may include a cloud computing system having hardware and software for hosting virtualized computing devices, including a virtual machine (VM) hosted within the cloud computing system.

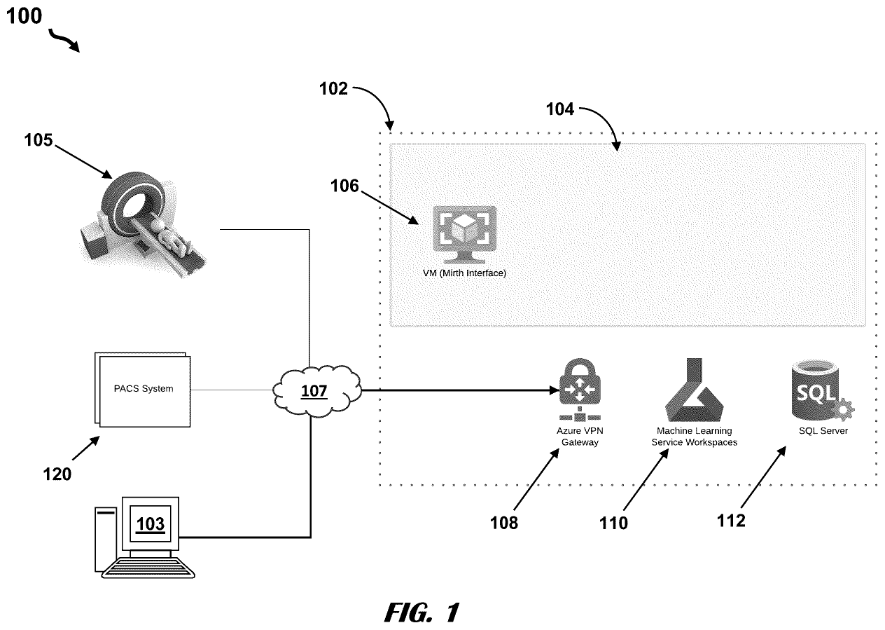

FIG. 1 shows components of a system 100 for generating MRI reports using machine-learning architectures. The system 100 includes a reporting system 102, client devices 103, medical imaging devices (e.g., MRI device 105), and a Picture Archiving and Communication System (PACS) 120. The components of the system 100, including the reporting system 102 and the PACS 120, may communicate with one another via one or more networks 107. The reporting system 102 includes a cloud computing system 104 having hardware and software for hosting virtualized computing devices, including a VM 106 hosted within the cloud computing system 104. The reporting system 102 may further include a Virtual Private Network (VPN) containing hardware and software components of the reporting system 102 and a VPN gateway 108 for remotely or securely accessing the components of the reporting system 102 via the one or more networks 107. The reporting system 102 further includes hardware and software components for executing an MRI report generation software (or report generator 110), which includes software programming of one or more machine-learning architectures. The reporting system 102 further includes a reporting system database 112 for storing various types of information related to generating the MRI reports, such as medical or image data records, operational logs, and medical illustrations, among other types of data. Embodiments may comprise additional or alternative components or omit certain components from what is shown in FIG. 1, yet still fall within the scope of this disclosure.

The one or more networks 107 may include various hardware and software components of one or more public or private networks for interconnecting the various components of the system 100. Non-limiting examples of such networks may include Local Area Network (LAN), Wireless Local Area Network (WLAN), Metropolitan Area Network (MAN), Wide Area Network (WAN), and the Internet. The communication over the network 107 may be performed in accordance with various communication protocols, such as Transmission Control Protocol and Internet Protocol (TCP/IP), User Datagram Protocol (UDP), and IEEE communication protocols. The VPN gateway 108 includes software programming for securely communicating with computing devices and software components that are internal or external to the reporting system 102 infrastructure. The operations of the VPN gateway 108 include, for example, encrypting and decrypting data packets sent or received via a VPN of the one or more networks 107.

The PACS 120 includes hardware and software components for storing various types of data. The PACS 120 generally includes a medical imaging technology for healthcare organizations to securely store, retrieve, distribute, and present medical images electronically, which may include storing medical images (e.g., MRI images, x-ray images) in a digital storage system. The PACS 120 includes an authorized, external computing system infrastructure for capturing, storing, and analyzing certain types of image data for the reporting system 102. For instance, the PACS 120 includes hardware and software components for communicating with the reporting system 102 and sending image data and reports to the reporting system 102 via the one or more networks 107, which may include a VPN and the VPN gateway 108.

In some embodiments, the reporting system 102 includes a cloud computing system 104 that includes various hardware and software components, such as hypervisors or virtualized computing resources, for hosting and executing various software applications or computing services of the reporting system 102. The cloud computing system 104 may include, for example, virtualized or “bare metal” instances of servers, routers, firewalls, databases, gateway devices, or other types of computing resources. In the example system 100, the reporting system 102 includes a VM 106 as computing device that executes certain operations for managing or handling the operations and interactions of the reporting system 102, though the operations and features of the VM 106 may be performed any form of computing device.

The VM 106 within the cloud computing system 104 (or other computing device) is used to execute various operations for interacting with or otherwise operating the report generator 110 and other functionality of the reporting system 102. The VM 106 of the reporting system 102 executes, for example, software programming for an interface engine (e.g., Mirth®) that handles receiving reports in a healthcare data messaging format, such as Health Level 7 (HL7) and image data from the MRI device 105 or the client device 103 of a care provider. The interface engine receives these HL7 messages containing various types of MRI image data or medical data and processes, such healthcare data to prepare the MRI reports using the machine-learning architecture of the report generator 110.

The interface engine includes software programming executed by the VM 106 or other computing device, such as the open-source Mirth® software programming. The interface engine is programmed or otherwise designed for healthcare applications, enabling the integration and exchange of clinical data between various components of the reporting system 102 (e.g., client devices 103, MRI device 105, VM 106, PACS 120). The interface engine of the VM 106 receives the incoming reports as HL7 messages from the PACS 120 or other data source, processes them, and creates a final report. The VM 106 then sends or returns the final report or other outputs back to the PACS 120 or other destination (e.g., VM 106) via the one or more networks 107.

In some embodiments, the VM 106 includes various logical communications and processing channels or pipelines for sending and receiving certain types of data or instructions via the one or more networks 107. The VM 106 may receive the HL7 messages and return an acknowledgement message, to and from the PACS 120 or other data source using a source channel, where the HL7 messages include input reports. The VM 106 receives the input reports in a text format according to the HL7 standard, where the VM 106 may receive image data (e.g., MRI imagery), among other types of data, in conjunction with the input message.

In a processing channel, the VM 106 executes operations that, for example, extract the text (or other types of information) from report text of the input report and generate a summary of the input report. The VM 106 may execute one or more machine-learning architectures when performing the operations of the data processing channel, such as executing a neural network architecture for extracting feature vectors in the report text or a large language model (LLM) for generating the text summary for the input report. In some implementations, the VM 106 outputs the device-generated summary and one or more medical illustrations as appended to the original input report. The VM 106 or other component of the reporting system 102 may execute or otherwise utilize various custom Java packages to fulfill the tasks described herein with respect to the processing channel.

In an output channel (sometimes referred to as a return channel), the programming of the interface engine executed by the VM 106 instructs the VM 106 to send the updated output report to the PACS 120 or other destination device (e.g., client device 103). The interface engine instructs the VM 106 to send the updated report back to the PACS 120 or other device and receives an acknowledgment from the PACS 120 or other device that confirms successful transmission.

The report generator 110 includes software programming of one or more machine-learning architectures for generating reports using input medical data, image data, and illustrations data. The medical data and image data may be received from, for example, the client devices 103, MRI device 105, or PACS 120. The report generator 110 may query the reporting system database 112 for particular illustrations relevant to a patient.

To add illustrations and summarize the MRI reports, the report generator 110 executes or otherwise accesses one or more generative machine-learning models (sometimes referred to as “generative AI” models), such as a GPT model (e.g., GPT-4). The generative AI model processes the report data and generates clear and concise summaries that are easier to understand for a layman.

The report generator 110 or other component of the reporting system 102 may receive an MRI report from the PACS 120 as an HL7 message. The software of the report generator 110 extracts the various types of report data from the report in the HL7 message. The report generator 110 converts the extracted report data to a text file having a word processing file format (e.g., DOCX, PDF, TXT, RTF). The report generator 110 may extract the text for processing. In some cases, for example, the report generator 110 extracts the text at or between certain bookmarks (e.g., at Section1; between StartSection1 and EndSection1) indicating portions of the text file for further processing.

When the report generator 110 has the text from the original HL7 message containing the MRI report, the trained machine-learning models of the machine-learning architecture of the report generator 110 may generate the text of the report summary for the output reports. The report generator 110 may further select and extract a set one or more selected medical illustrations from the reporting system database 112 in accordance with one or more anatomical or anatomy indicators for portions of human anatomy (e.g., a portion of the spine indicated in the MRI report or output report) and one or more conditions indicators (e.g., location of herniation, severity of herniation). For each particular illustration selected from the reporting system database 112, the report generator 110 may generate one or more visual overlay elements as a representation of the medical conditions indicated by the one or more one or more condition indicators of the MRI report or output report. The report generator 110 updates and outputs the particular illustration having the visual overlay in the output report, in addition to the text of the output report.

In operation, the report generator 110 selects appropriate illustrations for both Sagittal and Axial views based upon the inputted image data. For this, the report generator 110 executes various machine-learning architectures to extract, identify, or otherwise determine medical information or condition indicators using the medical information of the input report, such as a herniation level, size, and curvature type information, from the report text. The report generator 110 may later use this information as parameters to select appropriate, relevant illustration images from the reporting system database 112. In some cases, special prompts are crafted to make sure all the required information is extracted and/or avoid unwanted information, such as bulges.

The report generator 110 receives or generates an AI prompt for a sagittal view. For instance, herniation condition information for sagittal view, such as spine region, curvature type, herniated levels, and herniation sizes, may be obtained by sending the report text to the machine-learning model with one or more AI prompts to an LLM or GPT or similar machine-learning model of the machine-learning architecture of the report generator 110. An example AI prompt may include:

First review and understand the following radiology report. Then extract information from the given report and return the response in the following JSON format {“spine_region”: “L-Spine or T-Spine or C-Spine”, “curvature_type”: “Normal or Straight or Reverse”, herniated_levels: “List of herniated levels as comma separated string”, herniation_sizes: “List of herniation sizes as comma separated string corresponding to the same herniated level.”}}. IGNORE bulge, if no herniation is found then return empty strings.

The report generator 110 receives or generates an AI prompt for an axial view. For instance, herniation condition information for axial view, such as herniation direction, spine region, and herniation sizes, may be obtained by sending the report text to the machine-learning model with one or more AI prompts to the LLM or GPT or similar machine-learning model of the machine-learning architecture of the report generator 110. An example AI prompt may include:

First review and understand the following radiology report. Then extract information about only one single biggest herniation from the report and return the response in the following JSON format {“spine_region”: “L-Spine or T-Spine or C-Spine”, herniated_level: “Herniated level”, herniation_direction: “List of herniation direction for herniated level as comma separated string. Possible herniation directions are ‘Central’, ‘Left Central’, ‘Left Far Lateral’, ‘Left Neural Foraminal’, ‘Left Paracentral’, ‘Right Central’, ‘Right Far Lateral’, ‘Right Neural Foraminal’, ‘Right Paracentral’”, “herniation_size”: “Size of the biggest herniation.”}}. IGNORE bulge, if no herniation is found then return empty strings.

The report generator 110 may query the reporting system database 112 to identify and select a set of one or more illustration images. Once the report generator 110 has the information about herniation level, herniation direction, herniation sizes, curvature type, and spine region, the report generator 110 may use this information to identify and select the appropriate illustrations.

Optionally, before querying the reporting system database 112 for the illustration data, the report generator 110 or other component of the operation 202 confirms whether the report is about C-Spine or L-Spine. This is because the illustrations are added for these two types of reports. For this purpose, the report generator 110 checks a “spine_region” field value extracted for Sagittal and Axial views. If the value of the spine region is one of C-Spine or L-Spine, then the report generator 110 may perform certain operations.

The report generator 110 queries the reporting system database 112 using the extracted data to find the appropriate illustration based on values of Herniation Level/Direction, Sizes, and curvature type. If no matching illustration is found in the reporting system database 112, a default illustration is used. Otherwise, the report generator 110 appends the corresponding illustration the source report. In some implementations, each illustration image is placed on a VM 106, whereas the reporting system database 112 contains data indicating a full path of each image.

The extracted report text is sent to a generative machine-learning model of the report generator 110 for summarization. The report generator 110 may perform certain operations for generating a machine-generated summary text.

The report generator 110 may receive or generate an AI prompt for the summarization. An example AI prompt may include:

Simplify this radiology report for a layman. Format your response in XHTML 1.0 Strict DTD.

Because the outputted summary from the generative AI is often plain text, the report generator 110 may convert this text into a certain word processing text file format (e.g., DOCX, RTF, PDF) that is generally an easy-to-read and nice-looking format. In some cases, for this purpose, the prompt above directs the AI to get a response as XHTML that is later converted into the word processing text file format. For instance, the report generator 110 may convert the text of the summary from an XHTML summary to a certain word processing text file format (e.g., DOCX, RTF, TXT, PDF) by executing the XHTML Import for docx4j Java package.

In some implementations, the report generator 110 may merge the AI report summary text, the selected illustrations, and the input source report. The report generator 110 may, for example, append the summarized word processing text file and the illustration images to the source report in a word processing text file format (e.g., DOCX, RTF, TXT, PDF), thereby generating the enhanced output report.

The reporting system database 112 is hosted in non-transitory machine-readable storage of the reporting system 102. The reporting system database 112 stores various types of data, such medical data, image data, report data, transaction logs, and illustrations, among other types of data.

The reporting system database 112 may include illustration data related to illustrations that the report generator 110 may select for generating the outputted reports. For instance, the illustration data may include an illustrations table in the reporting system database 112 that contains possible combinations for sagittal and axial views. For a sagittal view, the illustrations table includes herniation levels, herniation sizes, spine regions, and curvature types. For the axial view, the illustrations table includes herniation directions and herniation sizes. When the VM 106 receives the response from the report generator 110 for either the sagittal or axial view, the VM 106 may query the illustrations table with the herniation information to retrieve the appropriate illustration. TABLE 1 shows an example structure of the illustrations table:

TABLE 1

| Column Name | Type |
|---|---|
| IllustrationID | int |
| FilePath | nvarchar(MAX) |
| SpienRegion | nvarchar(20) |
| ViewType | nvarchar(20) |
| CurvatureType | nvarchar(20) |
| HerniationLevels | nvarchar(MAX) |
| HerniationDirection | nvarchar(MAX) |
| HerniationSizes | nvarchar(MAX) |

In some implementations, the reporting system database 112 includes transaction logs for troubleshooting and maintaining logs of transactions and processes. For instance, the report generator 110 or other component of the system 100 (e.g., client devices 103, VM 106) references the reporting system database 112 (e.g., SQL Server) for diagnosing errors occurring in the reporting system 102.

The software of the VM 106 records various events and errors in one or more tables of the reporting system database 112 for auditing and logging purposes. Non-limiting example tables include an illustration results table (TABLE 2) and a logs table (TABLE 3).

The example structure of the illustration results table (TABLE 2) is shown below:

TABLE 2

| Column Name | Type |
|---|---|
| illustrationResultsId | bigint |
| accessionNumber | nvarchar(100) |
| reportNumber | nvarchar(100) |
| viewType | nvarchar(150) |
| spineRegion | nvarchar(150) |
| curvatureType | nvarchar(150) |
| HerniationLevels | nvarchar(MAX) |
| herniationDirections | nvarchar(MAX) |
| filePath | nvarchar(150) |
| HerniationSizes | nvarchar(MAX) |
| createdAt | datetime |

The example structure of the logs table (TABLE 3) is shown below:

TABLE 3

| Column Name | Type |
|---|---|
| LogID | int |
| AccessionNo | nvarchar(20) |
| MessageId | nvarchar(20) |
| LogType | nvarchar(50) |
| ExtractedReportText | nvarchar(MAX) |
| AIResponse | nvarchar(MAX) |
| isError | bit |
| ErrorDesc | nvarchar(MAX) |
| createdAt | datetime |

Whenever a component of the system 100 (e.g., report generator 110) executes a query on the illustrations table of the reporting system database 112 to obtain illustration images, report generator 110 and reporting system database 112 capture, store, and log the parameters into the illustration results table (TABLE 2) and/or logs table (TABLE 3) of the reporting system database 112. This indicates, and helps later identify, which illustration the report generator 110 retrieved and included in a report and what the requested parameters were. The results table may also include entries that indicate when the report generator 110 query did not find or identify the illustration image for the query requested parameters. In these cases, the report generator 110 may be configured to reference and include optional default illustrations stored in the reporting system database 112 or other device of the reporting system 102.

In some embodiments, a server or other computing devices in the reporting system 102 executes software programming of a webserver for hosting a website and web application (sometimes referred to as a “web app”), accessible to the client device 103 or other device of the system 100. The web app is programmed to query, retrieve, and display the data from the illustration results (TABLE 2) and logs table (TABLE 3), which may be user interface (e.g., web browser) or word processing file in a paginated format (e.g., DOCX, PDF, TXT, RTF). This functionality allows users to sort and filter the data directly from a web interface, eliminating the need to manually query the reporting system database 112.

Additionally or alternatively, the web app includes a “prompt playground” webpage where users can test LLM or GPT prompts against different LLM or GPT machine-learning models.

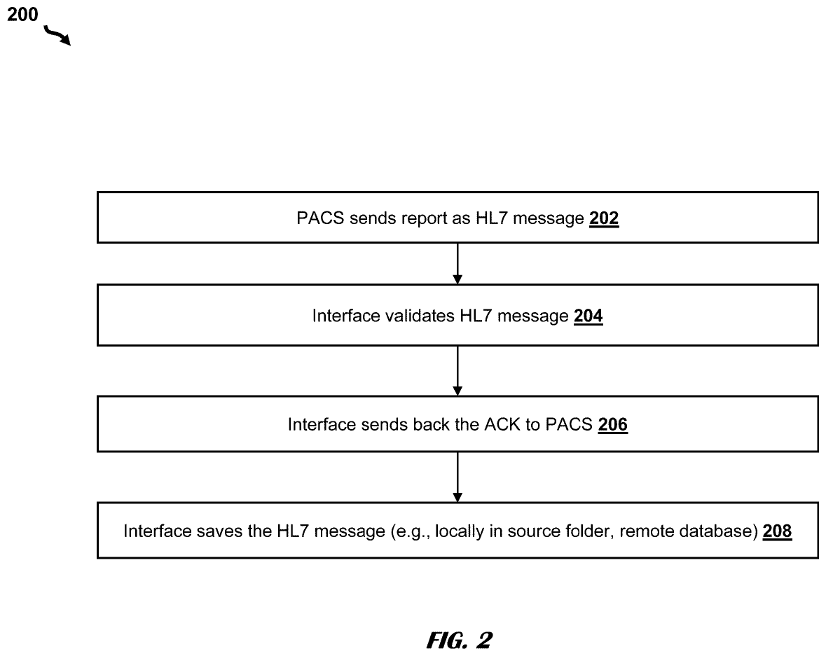

FIG. 2 shows operations of a process 200 for receiving medical image data and medical records data for machine-generated output reports using machine-learning architectures of a medical reporting system. As described in FIG. 2, the operations and features of the process 200 are executed by a computer (e.g., VM 106, server computer) of a medical data reporting system (e.g., reporting system 102), though embodiments are not so limited.

At operation 202, the computer receives an MRI report via one or more networks using software components of an interface engine for a source channel. The PACS or other data source sends the MRI report to the interface engine. The MRI report may be transmitted or received as a message structure in the HL7 message format standard. The source channel is set up to listen for these incoming messages and capture the MRI report at arrival. In some embodiments, the computer receives the MRI report as an HL7 message transmitted over a secure network connection, such as a VPN established via the VPN gateway 108. The computer may receive the MRI report from the PACS 120, the MRI device 105, or the client device 103 of a care provider. The HL7 message may include various segments containing patient demographic information, study metadata, and embedded medical imaging data or references to imaging data stored in a remote repository. In some cases, the source channel of the interface engine is configured to monitor a designated network port or message queue for incoming HL7 messages and to initiate processing operations upon detecting a new message arrival. The computer may log the receipt of the MRI report, including a timestamp, source identifier, and message identifier, to the reporting system database 112 for auditing and troubleshooting purposes.

At operation 204, the software source channel of the computer validates the data structure and content of the MRI report. For instance, the computer checks the structure and content of the HL7 message to confirm compliance with the HL7 messaging standard and internal formatting requirements. The computer may parse the HL7 message to extract individual segments and fields, verifying that required segments are present and that field values conform to expected data types and formats. In some embodiments, the computer applies schema validation operations to confirm that the HL7 message adheres to a predefined message schema, such as an HL7 v2.x or v3.x schema corresponding to the type of medical report being received. The computer may also perform content validation operations, such as checking that patient identifiers, accession numbers, and study dates are populated and formatted correctly. In some cases, the computer validates that the MRI report includes required clinical data, such as text report content or references to imaging data files. If the computer detects validation errors, the computer may generate an error log entry in the reporting system database 112 and may optionally send an error notification to the source system or the client device 103. If the validation is successful, the computer proceeds to the next operation.

At operation 206, once validated, the computer sends a confirmation or acknowledgment message back to the PACS system. The acknowledgment message may be formatted as an HL7 acknowledgment (ACK) message according to the HL7 messaging standard. The computer generates the acknowledgment message to indicate successful receipt and validation of the MRI report, and the computer transmits the acknowledgment message to the source system (e.g., PACS 120) via the one or more networks 107. In some embodiments, the acknowledgment message includes metadata such as a message identifier, a timestamp, and a status code indicating the validation result. The computer may log the transmission of the acknowledgment message to the reporting system database 112 for auditing and troubleshooting purposes. The acknowledgment message confirms to the source system that the MRI report has been successfully received and validated, allowing the source system to mark the report as delivered and to proceed with any subsequent workflow operations.

At operation 208, the computer saves the validated HL7 message, containing the MRI report, to a non-transitory machine-readable storage medium accessible to the computer, such as a database or local storage media of the computer. The computer may store the HL7 message in a designated directory folder or database table within the reporting system database 112 or in a local file system of the VM 106 or other computing device. In some embodiments, the computer indexes the stored HL7 message by patient identifier, accession number, and timestamp to facilitate efficient retrieval and processing in subsequent operations. The computer may also extract and separately store the embedded MRI imagery data or references to imaging data stored in a remote repository, such as the PACS 120. The computer then triggers the execution of software programming of a processing channel of the interface engine, initiating the next phase of operations for generating the machine-generated output report. In some cases, the computer generates a notification or event message to signal to the processing channel that a new MRI report is available for processing, and the processing channel retrieves the stored HL7 message from the storage location to begin extracting and analyzing the report content.

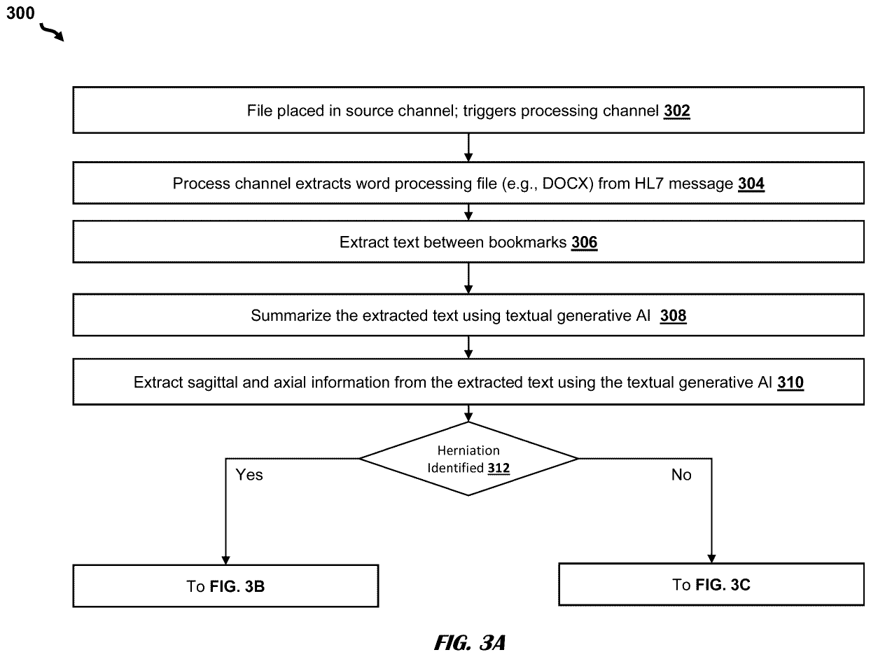

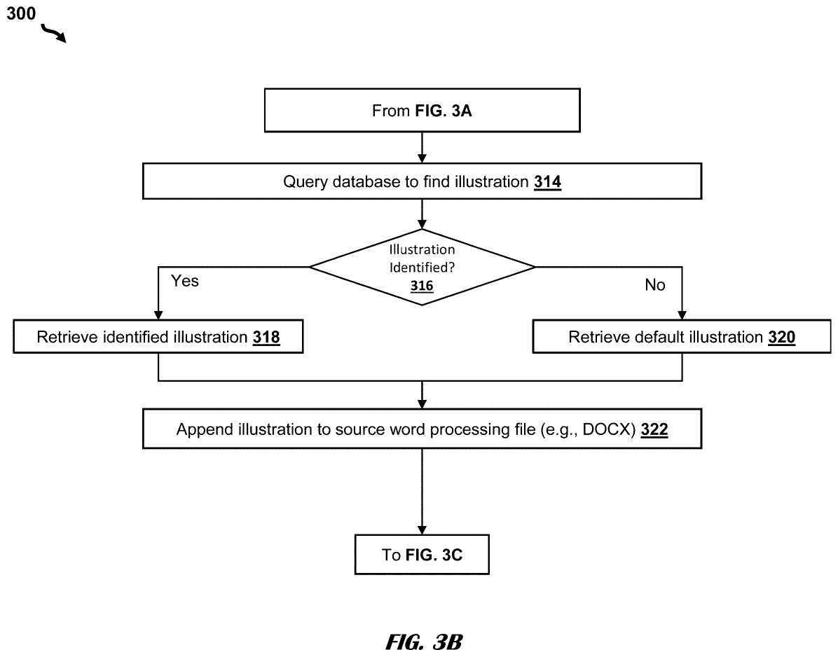

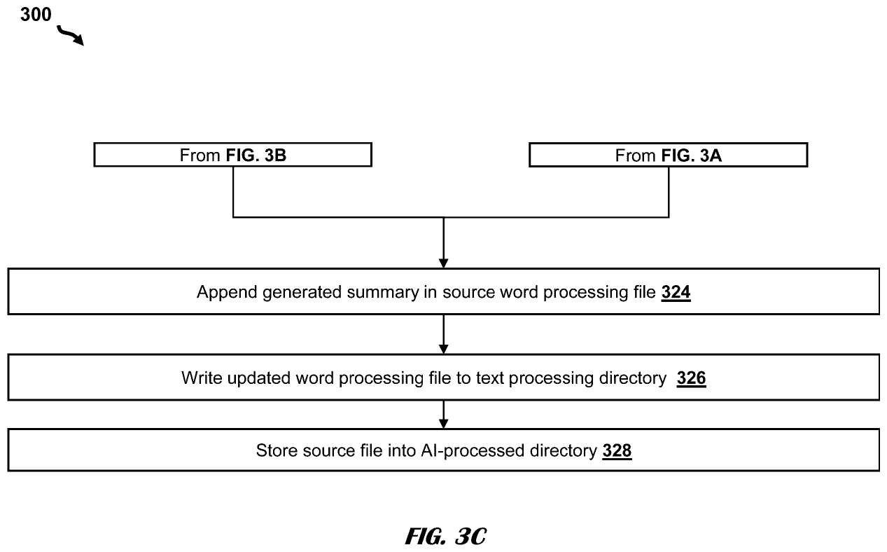

FIGS. 3A-3C show operations of a process 300 for executing machine-learning architectures of a medical reporting system trained for generating machine-generated output reports. The operations and features of the process 300 are executed by a computer (e.g., VM 106, server computer) of the medical data reporting system (e.g., reporting system 102), though embodiments are not so limited.

Turing to FIG. 3A, at operation 302, the computer saves and stores an HL7 message of an MRI report in a directory folder of a non-transitory storage location and executes software programming of a processing channel of an interface engine or other software program. In some implementations, the processing channel operations in the process 300 are triggered when the computer stores the HL7 message into the storage location. For example, the processing channel may monitor the directory folder for new file creation events, and may initiate extraction and analysis operations upon detecting that a new HL7 message file has been written to the directory folder. In some implementations, the computer may trigger the processing channel operations by generating an event message or inter-process communication signal that indicates the availability of a new HL7 message for processing. The event message can include metadata identifying the file path, file name, and/or a unique message identifier associated with the stored HL7 message, and the processing channel can retrieve the HL7 message from the storage location using the metadata included in the event message.

At operation 304, software programming of the processing channel executed by the computer extracts the report in word processing file format (e.g., DOCX file format) from the HL7 message. In some implementations, the computer extracts a DOCX-formatted file embedded within a designated segment of the HL7 message. For example, the computer can parse the HL7 message structure to locate a segment containing encoded document content, decode the content from a base64 encoding or other encoding scheme, and write the decoded content to a local storage location or buffer as a DOCX file accessible for subsequent text extraction operations. In some implementations, the computer extracts metadata from the HL7 message, including patient identifiers, accession numbers, or study timestamps, and associates the metadata with the extracted DOCX file to facilitate retrieval and correlation with corresponding MRI imagery data.

At operation 306, the computer extracts the text between specified markers in the text file. The specified markers can be bookmarks embedded in the word processing file that delineate a portion of the document containing report content intended for processing. For example, the computer can open the DOCX file, scan the document structure for bookmark elements labeled StartSection1 and EndSection1, and extract all text content occurring between the two bookmarks into a string or text buffer for further analysis. In some implementations, the computer extracts the text content by parsing the document object model of the word processing file, traversing paragraph nodes and text runs within the delimited region, and concatenating the text runs into a continuous text string while preserving formatting information such as line breaks or paragraph boundaries. The computer can discard portions of the document outside the bookmarked region, thereby isolating the relevant report text for subsequent summarization or descriptor extraction operations.

At operation 308, the computer generates a text summary by executing or accessing an LLM or similar machine-learning model of a generative AI software program. In some implementations, the computer transmits the extracted text to a third-party generative AI machine-learning model software instance, such as an Azure® OpenAI® LLM service, via an application programming interface. For example, the computer can construct a request message that includes the extracted text and a prompt string specifying that the text is to be simplified for a layman, and the computer can receive a response message containing the generated summary text formatted according to a markup language such as XHTML. The computer can apply the prompt string that instructs the LLM to format the response in XHTML 1.0 Strict DTD, and the LLM can generate the summary text embedded within valid XHTML tags that the computer can subsequently convert to a word processing file format. In some implementations, the computer executes a locally hosted LLM or similar generative machine-learning model, and the computer provides the extracted text as input to the locally hosted model and retrieves the generated summary text from the model output.

At operation 310, the computer extracts sagittal and axial information from the extracted text using the textual generative AI. In some cases, a radiologist or other user operates an end-user device to indicate or selects one or more key MRI slice from the patient's scan (in the MRI image report), where the selected slice indicates or highlights a primary area of concern (e.g., a herniation). Optionally, the computer or other device of the system colorizes the selected MRI slice(s), either manually or using generative AI, to emphasize critical features as according to the input report data, such as herniations and spinal curvature.

At operation 312, the computer determines or identifies a herniation or other medical condition in one or more condition indicators. The computer analyzes the extracted sagittal and axial information obtained from the machine-learning model (e.g., the generative AI or LLM) to identify whether a herniation or other medical condition is present in the patient's MRI report. The condition indicators may include, for example, a herniation level (e.g., C5-C6, L4-L5), a herniation size (e.g., small, medium, large, or a measured dimension in millimeters), a herniation direction (e.g., central, left paracentral, right neural foraminal), a curvature type (e.g., normal, straightening, reversal), and/or other anatomical or pathological descriptors extracted from the input source report text. The computer may parse the structured output generated by the generative AI or LLM (e.g., a JSON object containing key-value pairs corresponding to the extracted descriptors) to determine whether a herniation is identified. In some embodiments, the computer checks whether the structured output contains non-empty values for fields corresponding to herniation levels, herniation sizes, or herniation directions. For example, the computer may evaluate whether a “herniated_levels” field in the JSON output contains a comma-separated list of vertebral levels or whether the field is an empty string, and the computer may similarly evaluate whether a “herniation_sizes” field contains size descriptors or remains empty.

If the computer determines that the structured output contains one or more non-empty condition indicators corresponding to a herniation or other medical condition, the computer proceeds to operation 314 of FIG. 3B to query the illustration database for a relevant medical illustration image matching the identified condition indicators. If the computer determines that the structured output does not contain any condition indicators corresponding to a herniation or other medical condition (e.g., all herniation-related fields are empty strings), the computer proceeds to operation 324 of FIG. 3C to append the AI-generated summary text to the source report without adding a medical illustration image corresponding to a herniation. In some implementations, the computer may log the determination result to the reporting system database 112 for auditing and troubleshooting purposes, including a record indicating whether a herniation was identified and which condition indicators were extracted from the input source report.

Turning to FIG. 3B, at operation 314, the computer queries a database containing medical illustrations. The computer may query the illustration database (e.g., reporting system database 112) to identify and retrieve one or more medical illustration images that match anatomical descriptors and condition indicators extracted from the input source report. In some embodiments, the computer constructs a database query using the extracted key descriptors, indicators, or parameters from the input source radiology report, including herniation levels (e.g., C5-C6, L4-L5), herniation sizes (e.g., small, medium, large, or measured dimensions in millimeters), herniation directions (e.g., central, left paracentral, right neural foraminal), curvature types (e.g., normal, straightening, reversal), and spine regions (e.g., C-Spine, L-Spine, T-Spine). The computer may execute the database query against an illustrations table that stores metadata associated with a plurality of medical illustration image files, where each medical illustration image file includes attributes corresponding to anatomical descriptors and condition indicators. For example, the computer may query the illustrations table for medical illustration images having a “spine_region” attribute matching “C-Spine” and a “herniation_levels” attribute matching “C5-C6” and a “herniation_sizes” attribute matching “medium” and a “curvature_type” attribute matching “reversal.”

In some implementations, the computer applies matching operations that compare the extracted descriptors against the metadata attributes stored in the illustrations table, and the computer selects one or more medical illustration images where the metadata attributes satisfy a matching criterion (e.g., exact match, partial match, or similarity threshold). The computer may also apply ranking operations to prioritize medical illustration images that most closely match the extracted descriptors, for example by assigning a match score to each candidate medical illustration image based on the number of matching attributes or the degree of similarity between the extracted descriptors and the metadata attributes. In some cases, the computer may filter the query results to exclude medical illustration images that do not satisfy minimum matching requirements, such as requiring that at least the spine region and herniation level attributes match the extracted descriptors. The computer may also apply domain-specific filtering operations, for example by limiting the query to medical illustration images corresponding to C-Spine or L-Spine regions when the input source report indicates a cervical or lumbar spine study. The database query operations may be executed during the processing channel operations of the interface engine, and the query results may be stored in a temporary buffer or cache for subsequent retrieval and processing in operation 316.

At operation 316, the computer determines whether the query successfully identified the one or more relevant medical illustration images in the database. The computer evaluates the query results obtained in operation 314 to determine whether at least one medical illustration image satisfies the matching criteria based on the extracted descriptors from the input source report. In some embodiments, the computer checks whether the query results contain at least one medical illustration image file having metadata attributes that match the extracted anatomical descriptors and condition indicators. For example, the computer may evaluate whether the query results include at least one medical illustration image file where the “spine_region” attribute matches the extracted spine region, the “herniation_levels” attribute matches the extracted herniation level, and the “herniation_sizes” attribute matches the extracted herniation size.

In some implementations, the computer determines that the query successfully identified one or more relevant medical illustration images when the query results contain at least one medical illustration image file meeting a minimum match threshold, such as requiring that at least three out of four metadata attributes match the extracted descriptors. The computer may also reference and compare a match score for the query results against a match score threshold to determine whether the query results contain a medical illustration image that meets a minimum quality or relevance standard, for example by comparing the match scores assigned to candidate medical illustration images against a predefined threshold value.

If the computer determines that the query results contain at least one medical illustration image that satisfies the matching criteria, the computer proceeds to operation 318 to retrieve the identified medical illustration image. If the computer determines that the query results do not contain any medical illustration images that satisfy the matching criteria, the computer proceeds to operation 320 to retrieve a default medical illustration image. In some cases, the computer may log the determination result to the reporting system database 112 for auditing or review functions, including a record indicating whether a matching medical illustration image was identified and which metadata attributes were used in the matching operations.

At operation 318, the computer selects and retrieves the selected medical illustration images from the database. The computer retrieves one or more medical illustration image files identified in operation 316 that match the anatomical descriptors and condition indicators extracted from the input source report. In some embodiments, the computer retrieves the medical illustration image files from the illustration database by accessing a file path attribute stored in the illustrations table, where the file path attribute indicates a storage location of the medical illustration image file in a local file system or remote storage system. For example, the computer may retrieve a medical illustration image file stored on the VM 106 or in a cloud storage service accessible via the one or more networks 107. The computer may retrieve the medical illustration image file by retrieving or reading the file content from the storage location and loading the file content into a temporary buffer or cache for subsequent processing and insertion into the output report.

In some implementations, the computer selects the medical illustration image file having the highest match score or the most closely matching metadata attributes when multiple medical illustration images satisfy the matching criteria. The computer may also retrieve multiple medical illustration image files when the extracted descriptors indicate multiple herniation levels or multiple anatomical regions of interest, for example by retrieving a first medical illustration image for a sagittal view and a second medical illustration image for an axial view. The computer may apply formatting operations to the retrieved medical illustration image files, such as resizing, cropping, or annotating the medical illustration images to conform to output report formatting requirements. In some cases, the computer stores the retrieved medical illustration image files in a temporary directory or cache location for later insertion into the output report during the merge or append operations described in operation 322.

Optionally, at operation 320, the computer may return one or more default medical illustration images when the computer does not identify the relevant medical illustration images in the database. The computer retrieves one or more default medical illustration image files from the illustration database when the query results obtained in operation 314 do not contain any medical illustration images that satisfy the matching criteria evaluated in operation 316. In some embodiments, the computer retrieves a predefined default medical illustration image file corresponding to a generic or placeholder illustration for the spine region indicated in the input source report, such as a default C-Spine illustration or a default L-Spine illustration. The default medical illustration image file may depict a normal or typical anatomical configuration without specific herniation or condition indicators, and the default medical illustration image file may include generic labels or annotations indicating the spine region and anatomical structures.

As an example, according to operations 314-320, the computer executes a generative AI (or other type of machine-learning model) to generate or update an illustration image based upon the patient's particular condition. The computer determines or selects the medical illustrations according to the key descriptors, indicators, or parameters as in the condition indicators for the patient's condition. Using anatomy indicators and/or condition indicators in the source report, the computer may generate the output illustration using an illustration selected from the database or, alternatively, generate the output illustration using a default illustration selected from the database.

At operation 322, the computer merges or appends the selected relevant illustrations to a new or updated version of the original report in the text file. The computer retrieves the medical illustration image files (as in operation 318) or the default medical illustration image files (as retrieved in operation 320), and the computer integrates the medical illustration image files into the source word processing file containing the input source report text. In some embodiments, the computer appends the medical illustration image files to the source word processing file by inserting the medical illustration images at designated locations within the document structure, such as at the end of the document, after or interstitial within the report text, such designated sections corresponding to anatomical regions or condition indicators. The computer may apply formatting operations to the medical illustration image files to conform to output report formatting requirements, such as resizing the medical illustration images to fit within page margins, adjusting image resolution or quality settings, or applying captions or labels to the medical illustration images that identify the anatomical region, view type (e.g., sagittal, axial), or condition indicators (e.g., herniation level, herniation size) associated with the medical illustration image.

In some implementations, the computer modifies the source word processing file by programmatically accessing the document object model of the word processing file, locating designated insertion points or bookmarks within the document structure, and inserting image elements corresponding to the medical illustration image files at the insertion points. The computer may embed the medical illustration image files directly into the word processing file as inline image elements, or the computer may insert references or links to the medical illustration image files stored in external storage locations. The computer may also insert descriptive text or annotation content adjacent to the medical illustration images to provide explanatory information regarding the anatomical features or condition indicators depicted in the medical illustration images. For example, the computer may insert text labels identifying vertebral levels, herniation directions, or curvature types illustrated in the medical illustration images, or the computer may insert captions referencing the input source report text that describes the corresponding medical condition.

In some cases, the computer generates a new or updated version of the source word processing file by creating a copy of the original source word processing file and appending the medical illustration image files to the copy, thereby preserving the original source word processing file in an unmodified state. The computer may store the new or updated version of the source word processing file in a designated directory folder or database table within the reporting system database 112 or in a local file system of the VM 106 or other computing device. The computer may index the new or updated version of the source word processing file by patient identifier, accession number, and timestamp to facilitate efficient retrieval and processing in subsequent operations.

Turning to FIG. 3C, at operation 324, the computer merges or appends the AI-generated summary text the new or updated version of the original report in the text file having the relevant illustrations selected from the database. The computer may generate or otherwise output an enhanced output report based on the source text file and illustrations, as generated or selected using the source MRI report.

The computer may, for example, execute generative AI operations that combines the colorized MRI image with the selected illustration, overlaying anatomical details from the MRI onto the illustration and vice versa. In some implementations, the computer executes operations for selecting a most significant axial image depicting a comparatively largest herniation and a direction of the largest herniation. For axial images and sagittal images, the computer may implement similar operations or processes (e.g., generative AI operations) for colorizing and merging the axial or sagittal images with the relevant illustrations.

In some embodiments, the generative AI operations refine the combination through multiple iterations, creating two versions: one that emphasizes the illustration with MRI anatomical details, and another that emphasizes the MRI with illustrative elements. In some implementations, the computer may execute generative AI operations that recursively enhance images to balance anatomical accuracy and illustrative clarity. This process involves transformations to ensure both images retain their respective details while improving interpretability. In some implementations, the AI places the refined images on a graph to determine the most appropriate representations for inclusion in the final report. The computer selects the images that best balance anatomical accuracy and illustrative clarity, enhancing the understanding of radiology findings.

Optionally, at operation 326, the computer saves the enhanced report in a specific directory folder (e.g., processing-doc-files). At operation 328, the computer moves the original report file to another directory folder (e.g., ai-processed).

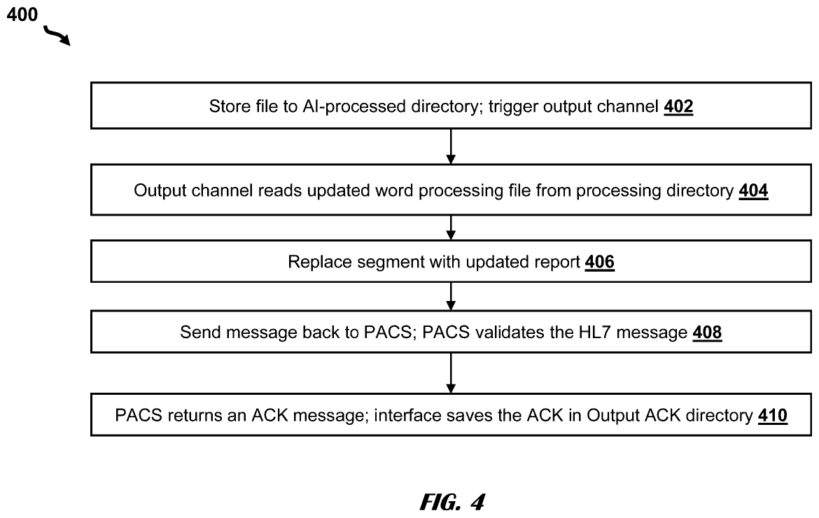

FIG. 4 shows operations of a process 400 for outputting medical image data and medical records data for machine-generated output reports using machine-learning architectures of a medical reporting system. As described in FIG. 4, the operations and features of the process 200 are executed by a computer (e.g., VM 106, server computer) of a medical data reporting system (e.g., reporting system 102), though embodiments are not so limited.

At operation 402, the computer saves and stores the final, enhanced output report into a directory folder (e.g., processing-doc-files folder) of a non-transitory storage location and executes software programming of an output channel (sometime referred to as a return channel) of an interface engine or other software program. In some cases, the output channel operations in the process 400 are triggered when the computer stores the output report into the storage location.

At operation 404, the software programming of the output channel executed by the computer retrieves or reads the enhanced output report from the storage location (e.g., processing-doc-files folder). The computer accesses the storage location by referencing a file path or directory identifier associated with the processing directory, and the computer reads the file content of the enhanced output report into memory or into a temporary buffer for subsequent processing operations. In some embodiments, the computer retrieves the enhanced output report by executing file system operations that locate the file based on a file name, accession number, or unique message identifier stored in metadata associated with the output report. The computer may verify the integrity of the retrieved file by checking file size, file format, or a checksum value to confirm the file has not been corrupted during storage operations.

In some implementations, the computer parses the enhanced output report to extract the updated report content, including the AI-generated summary text and the appended medical illustration images. The computer may also extract metadata from the enhanced output report, such as patient identifiers, accession numbers, and timestamps, to facilitate correlation with the original HL7 message received in operation 202. The computer may apply formatting operations to the enhanced output report content to conform to output formatting requirements specified by the destination system (e.g., PACS 120), such as converting the word processing file format to a standardized healthcare document format or embedding the report content within a designated segment of an HL7 message structure.

In some cases, the computer validates the structure and content of the enhanced output report to confirm the report includes required elements such as the AI-generated summary and at least one medical illustration image. The computer may also apply quality control operations, such as verifying the presence of required metadata fields, checking the file size or image resolution of appended medical illustration images, or confirming the report content conforms to predefined content standards or regulatory requirements. If the computer detects validation errors or quality control issues during retrieval operations, the computer may generate an error log entry in the reporting system database 112 and may optionally trigger a retry operation or send a notification to an administrator or user device.

At operation 406, the computer updates the original HL7 message with the updated enhanced report content. In some cases, the computer replaces a segment or portion of the original HL7 message with the updated enhanced report. The computer modifies the HL7 message structure by identifying a designated segment that contains the original radiology report content, such as an OBX (Observation/Result) segment or an embedded document segment, and replacing the segment content with the enhanced report content retrieved from the processing directory in operation 404. In some embodiments, the computer encodes the enhanced report content according to the HL7 messaging standard, for example by converting the word processing file format (e.g., DOCX) to a base64-encoded string or other encoding format compatible with HL7 segment field constraints. The computer may also update metadata fields in the HL7 message to reflect the modifications made to the report content, such as updating a timestamp field, a message type identifier, or a version number to indicate the report has been processed and enhanced by the machine-learning architecture of the reporting system 102.

In some implementations, the computer validates the modified HL7 message structure to confirm the message conforms to the HL7 messaging standard and includes all required segments and fields. The computer may apply schema validation operations to verify the message adheres to a predefined HL7 message schema (e.g., HL7 v.2, HL7 v.3) corresponding to the radiology report data. The computer may also verify the encoded enhanced report content does not exceed field length constraints imposed by the HL7 standard or by the destination system (e.g., PACS 120), and the computer may apply compression or truncation operations if necessary to conform to field length requirements.

In some cases, the computer generates a new message identifier or updates an existing message identifier to distinguish the modified HL7 message from the original HL7 message (as received in operation 202). The modified HL7 message is then prepared for transmission back to the PACS 120 or other destination device in operation 408.

At operation 408, the computer sends and returns the updated HL7 message back to the PACS or other source device. Optionally, software programming executed at a device of the PACS checks the updated HL7 message to validate accuracy and format.

At operation 410, the computer receives, via the output channel, a final acknowledgment message from a device of the PACS. The computer saves the final acknowledgement into an output acknowledgement directory folder in the storage location.

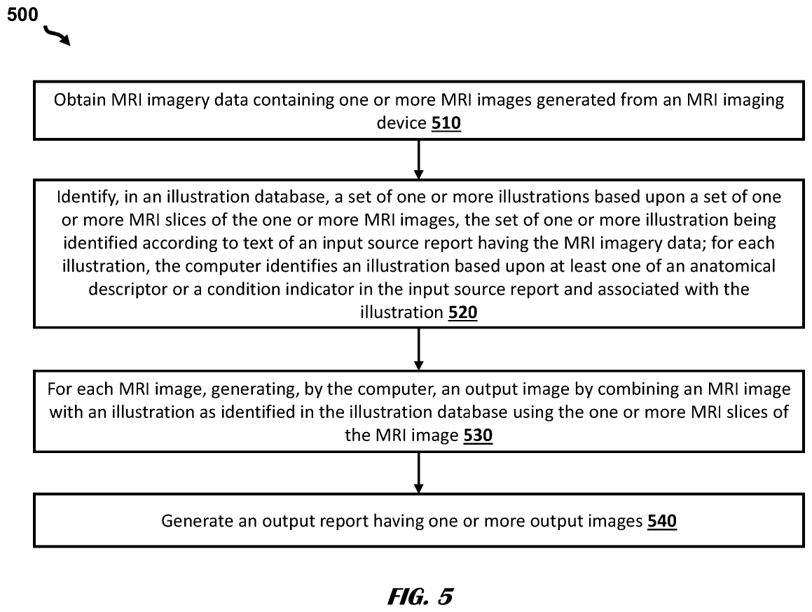

FIG. 5 shows operations of a process 500 for a computer-implemented method for enhancing radiology reports having medical imaging data. The operations and features of the process 500 are executed by a computer (e.g., VM 106, server computer) of the medical data reporting system (e.g., reporting system 102), though embodiments are not so limited.

At operation 510, the computer obtains MRI imagery data containing one or more MRI images generated from an MRI imaging device. The MRI imagery data may be obtained from the MRI imaging device directly via a network connection, from a PACS system storing previously captured MRI images, or from a client device operated by a healthcare provider. In some embodiments, the computer receives the MRI imagery data as part of an HL7 message transmitted over one or more networks, where the HL7 message includes embedded MRI image data or references to MRI image files stored in a remote repository. The computer may extract the MRI imagery data from the HL7 message by parsing the message structure, locating segments containing encoded image data, and decoding the image data from a base64 encoding or other encoding scheme. The computer may also receive metadata associated with the MRI imagery data, including patient identifiers, accession numbers, study timestamps, imaging sequence parameters, and anatomical region indicators. In some implementations, the computer validates the received MRI imagery data by checking file format compatibility, image resolution, and completeness of image sequences. The computer may store the received MRI imagery data in a local storage location or database for subsequent processing operations.

At operation 520, the computer identifies, in an illustration database, a set of one or more medical illustration images based upon a set of one or more MRI slices of the one or more MRI images. The computer identifies the set of one or more medical illustration images according to text of an input source report having the MRI imagery data. The input source report includes textual report content generated by a radiologist or other healthcare provider describing findings observed in the MRI images, and the textual report content may include anatomical descriptors and condition indicators corresponding to portions of human anatomy and medical conditions observed in the MRI imagery data. For each medical illustration image, the computer identifies a medical illustration image based upon at least one of an anatomical descriptor or a condition indicator in the input source report and associated with the medical illustration image. The anatomical descriptors may include, for example, spine region identifiers (e.g., C-Spine, L-Spine, T-Spine), vertebral level identifiers (e.g., C5-C6, L4-L5), and anatomical orientation descriptors (e.g., sagittal view, axial view). The condition indicators may include, for example, herniation level descriptors (e.g., C5-C6 herniation), herniation size descriptors (e.g., small, medium, large, or measured dimensions in millimeters), herniation direction descriptors (e.g., central, left paracentral, right neural foraminal), and curvature type descriptors (e.g., normal, straightening, reversal).

In some embodiments, the computer extracts the anatomical descriptors and condition indicators from the input source report text by executing a machine-learning model configured to parse radiology report text and identify key descriptors corresponding to anatomical features and pathological conditions. For example, the computer may apply a generative AI model, such as a large language model or a natural language processing model, to the input source report text using one or more prompt strings that instruct the model to extract specific types of descriptors and return the extracted descriptors in a structured format, such as a JSON object containing key-value pairs corresponding to the anatomical descriptors and condition indicators. The computer may execute a first prompt to extract sagittal view information, including spine region, curvature type, herniated levels, and herniation sizes, and the computer may execute a second prompt to extract axial view information, including spine region, herniated level, herniation direction, and herniation size. The computer may then use the extracted anatomical descriptors and condition indicators to query the illustration database.

The illustration database includes a plurality of medical illustration image files, where each medical illustration image file includes metadata attributes corresponding to anatomical descriptors and condition indicators. The computer constructs a database query using the extracted anatomical descriptors and condition indicators to identify one or more medical illustration image files having metadata attributes that match the extracted descriptors. The computer may apply matching operations that compare the extracted descriptors against the metadata attributes stored in the illustration database, and the computer may select one or more medical illustration images where the metadata attributes satisfy a matching criterion, such as an exact match, partial match, or similarity threshold. In some implementations, the computer applies ranking operations to prioritize medical illustration images that most closely match the extracted descriptors, for example by assigning a match score to each candidate medical illustration image based on the number of matching attributes or the degree of similarity between the extracted descriptors and the metadata attributes. The computer may retrieve the identified medical illustration images from the illustration database by accessing a file path attribute stored in the database that indicates a storage location of the medical illustration image file. In some cases, when the computer does not identify any medical illustration images satisfying the matching criteria, the computer retrieves a default medical illustration image corresponding to a generic or placeholder illustration for the spine region indicated in the input source report.

At operation 530, for each MRI image, the computer generates an output image by combining or merging an MRI image with the medical illustration image as identified in the illustration database using the one or more MRI slices of the MRI image. The computer combines or merges the MRI image and the medical illustration image by generating a composite image that includes visual elements from both the MRI image and the medical illustration image. In some embodiments, the computer generates the output image by overlaying anatomical details from the MRI image onto the medical illustration image, and/or by overlaying illustrative elements from the medical illustration image onto the MRI image. The computer may apply image processing operations, such as colorization, segmentation, contour detection, and blending, to integrate the MRI image and the medical illustration image into a cohesive visual representation that highlights anatomical features and medical conditions observed in the MRI imagery data.

In some implementations, the computer selects one or more MRI slices from the MRI imagery data to use as a basis for generating the output image. The computer may select the MRI slices based on user input received from a user device, where the user input indicates one or more key MRI slices that highlight a primary area of concern, such as a herniation or other anatomical abnormality. The computer may also select the MRI slices automatically by applying image analysis operations to the MRI imagery data to identify slices containing anatomical features or condition indicators corresponding to the descriptors extracted from the input source report. For each selected MRI slice, the computer may generate a colorized instance of the MRI slice by identifying a portion of the anatomy in the MRI slice having a medical condition according to the condition indicator of the input source report, and updating a coloring of the portion of the anatomy having the medical condition to emphasize the medical condition in the output image. The computer may apply generative AI operations to automatically colorize the MRI slice based on the condition indicators, or the computer may receive user input indicating manual colorization of specific anatomical regions in the MRI slice.

The computer generates the output image by merging the colorized MRI slice with the medical illustration image identified in operation 520. The computer may apply blending operations, opacity adjustments, or layer compositing operations to combine the MRI image and the medical illustration image in a manner that preserves anatomical accuracy while improving illustrative clarity. In some embodiments, the computer generates multiple versions of the output image, including a first version that emphasizes the medical illustration image with MRI anatomical details overlaid, and a second version that emphasizes the MRI image with illustrative elements overlaid. The computer may iteratively refine the output image through multiple processing iterations by applying transformations that balance anatomical accuracy and illustrative clarity, for example by adjusting color intensity, contrast, contour sharpness, or overlay transparency in successive iterations. In some implementations, the computer generates a refined image according to a graph representation of one or more image features, where the graph representation includes an anatomical accuracy value and an illustration clarity value for the medical illustration image, and the computer selects the output image that best balances the anatomical accuracy value and the illustration clarity value based on graph-based selection criteria.

At operation 540, the computer generates an output report having one or more output images and other components. The output report includes the one or more output images (as generated in operation 530), where each output image is a composite image combining an MRI image and a medical illustration image identified based on anatomical descriptors and condition indicators extracted from the input source report.

In some embodiments, the output report also includes a machine-generated text summary generated by a machine-learning model of a generative AI program based upon the text of the input source report. The computer may generate the machine-generated text summary by applying a large language model or other generative AI model to the input source report text using a prompt string that instructs the model to simplify the radiology report text for a layperson and format the response in a structured text format, such as XHTML or HTML. The computer may convert the machine-generated text summary from the structured text format to a word processing file format, such as DOCX, RTF, or PDF, and the computer may append the machine-generated text summary and the one or more output images to the input source report to generate the enhanced output report.

In some implementations, the computer transmits the output report to a destination device, such as a PACS system, a client device operated by a healthcare provider, or an end-user device operated by a patient. The computer may transmit the output report by updating an HL7 message with the enhanced report content and transmitting the updated HL7 message to the destination device via one or more networks. The computer may also store the output report in a database or local storage location for subsequent retrieval and distribution. The output report provides an enhanced radiology report that includes visual representations of medical conditions observed in the MRI imagery data, improving the interpretability of the radiology report for both medical professionals and patients by combining technical MRI imaging data with accessible medical illustrations and simplified explanatory text.

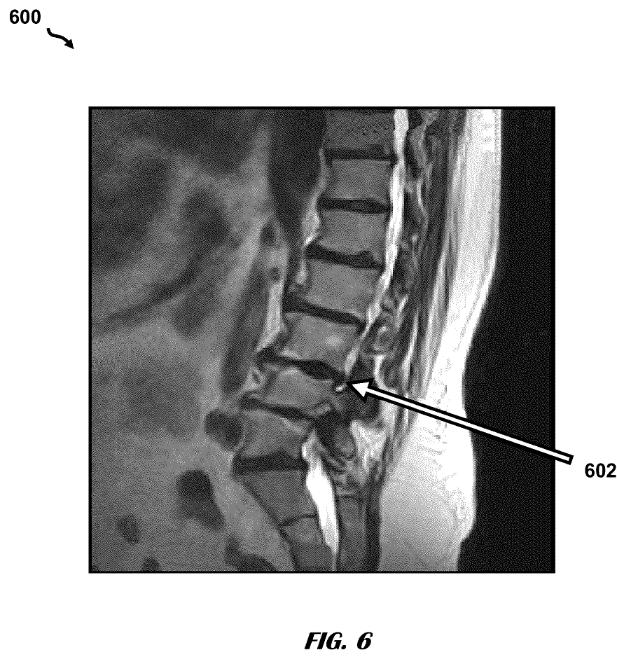

FIGS. 6-11 depict a series of graphical user interfaces 600-1100 displaying portions of a source report and an output enhanced report generated by one or more machine-learning models of a machine-learning architecture.

The source report pertains to lower back pain following a patient's injury (e.g., motor vehicle accident). The definitions of the input source report and/or output report may be based on preconfigured definitions of disc bulge, disc herniation, protrusion, and extrusion, which a computing device uses for training machine-learning models of the machine-learning architecture, such as a text-generating LLM. The definitions may be pulled from one or more training corpora of medical data and information, such as medical encyclopedias and medical journals, among others.

FIG. 6 depicts a sagittal view of a portion of the anatomy (e.g., T2 L3, L4). For instance, the graphical user interface 600 shows that at L3-L4 602, there is a right paracentral disc herniation superimposed on a disc bulge, the combination of which measures 5 mm indenting the ventral thecal sac and elevating the posterior longitudinal ligament; there are anterior and there are posterior osteophytes; the disc material extends posterior to the posterior osteophytes; there is a zone of hyperintensity within the disc consistent with an annular fissure; there is moderate right neural foraminal narrowing; there is mild left neural foraminal narrowing; there is moderate spinal stenosis to 0.8 cm; and there is narrowing of the right lateral recess and contact to the right L4 transiting nerve root.

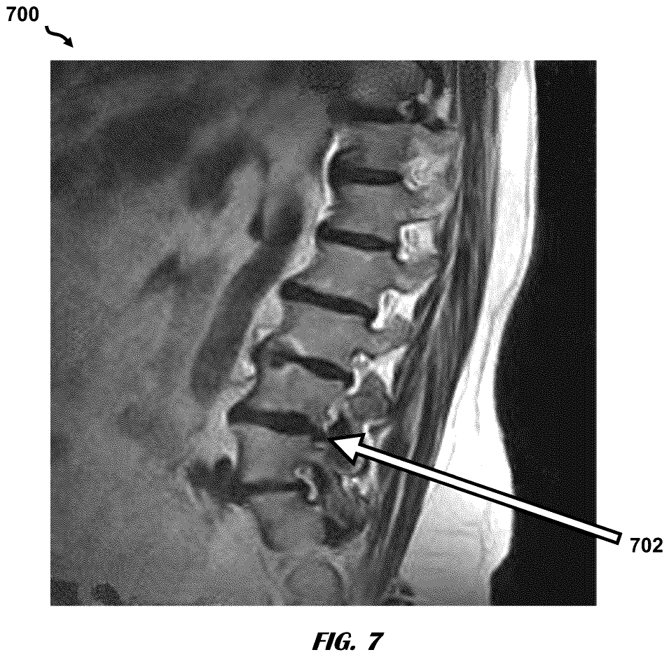

FIG. 7 depicts a sagittal view of a portion of the anatomy (e.g., T2 L4, L5). For instance, the graphical user interface 700 shows that at L4-L5 702, there is a left paracentral/neural foraminal disc herniation superimposed on a disc bulge, the combination of which measures 4.5 mm indenting the ventral thecal sac and elevating the posterior longitudinal ligament; there are anterior and there are posterior osteophytes; however, the disc material extends posterior to the posterior osteophytes; there is a zone of hyperintensity within the disc consistent with an annular fissure; there is severe bilateral neural foraminal narrowing; there is a 2.5 mm grade 1 retrolisthesis of L4 on L5; there is moderate spinal stenosis to 0.8 cm; and there is narrowing of the lateral recesses bilaterally.

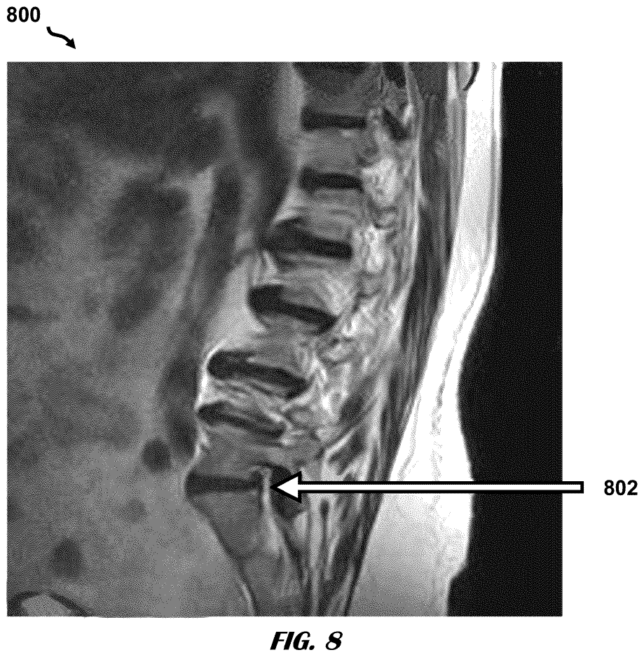

FIG. 8 depicts a sagittal view of a portion of the anatomy (e.g., T2 L5, S1). For instance, the graphical user interface 800 shows that at L5-S1 802, there are biforaminal disc herniations superimposed on a disc bulge, the combination of which measures 4 mm indenting the ventral thecal sac and elevating the posterior longitudinal ligament; there are anterior and there are posterior osteophytes; the disc material extends posterior to the posterior osteophytes; this is superimposed on a 2 mm grade 1 anterolisthesis of L5 on S1; there is moderate spinal stenosis to 0.7 cm; and there is severe bilateral neural foraminal narrowing.

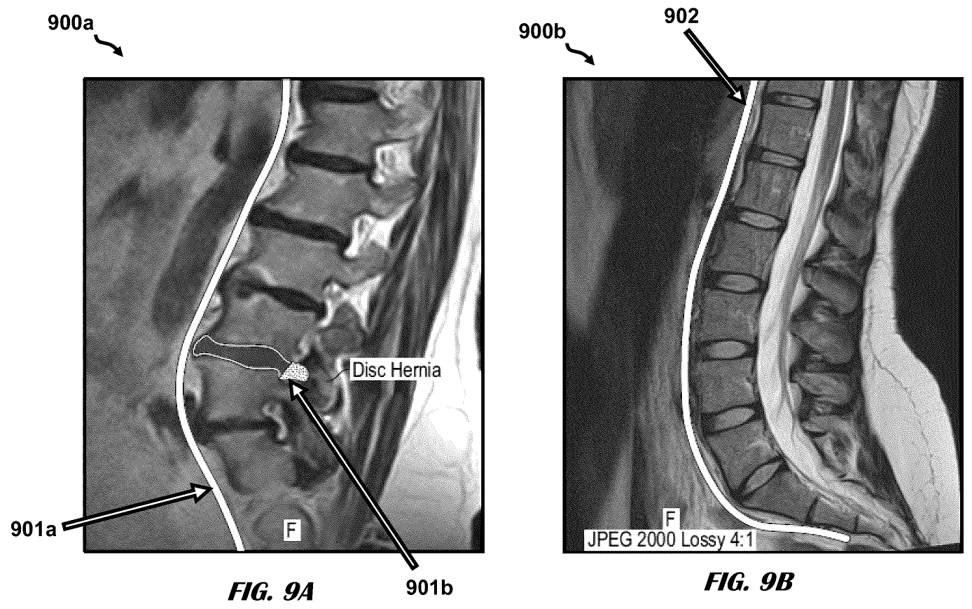

FIGS. 9A-9B depict the graphical user interfaces 900a, 900b having machine-generated overlays for an enhanced version of the MRI images. In FIG. 9A, the graphical user interface 900a displays the sagittal view of the portion of the anatomy (e.g., T2 L3, L4) in the graphical user interface 600 of FIG. 6 having the right paracentral disc herniation superimposed on the disc bulge. In the graphical user interface 900a of FIG. 9A, the software programming of a generative AI (or other type of machine-learning model) generates the one or more bolded or colorized overlays 901a-901b for portions of the anatomy or conditions. As an example, the generative AI may generate an anatomy overlay 901a that follows and indicates contours of the anatomy (e.g., interior portion of the spinal column). As another example, the generative AI may further generate a condition overlay 901b following and indicating contours of the medical condition (e.g., disc herniation).

For comparison, FIG. 9B depicts the graphical user interface 900b displaying the sagittal view of the comparable portion of the anatomy for a normal spine, without the herniation or other condition. In this graphical user interface 900b, the programming of the generative AI (or other type of machine-learning model) generates one or more bolded or colorized overlays, such as an anatomy overlay 902 that, for example, follows and indicates contours of the anatomy (e.g., the interior portion of the normal spinal column).

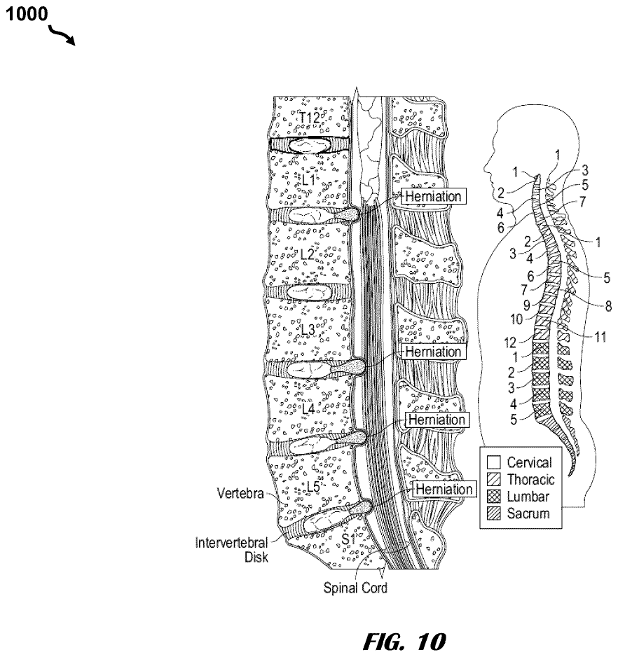

FIG. 10 depicts the graphical user interface 1000 displaying a machine-generated illustration for a sagittal view (side view) of the spine. The illustration image selection may be generated by the software programming of the generative AI executed by the computing device. The illustration includes additional image elements of portions of the anatomy, medical conditions, and annotations indicating some of the areas of interest as described in the input report (e.g., input radiology report). The generative AI may generate the illustration image according to anatomy indicators and/or condition indicators, where the generated illustration contains elements that depict or indicate a location of anatomy (e.g., vertebra, disk, spinal cord) or conditions and a severity of the condition (e.g., herniation). The generative AI generates the elements of the illustration of the graphical user interface 1000 based upon, for example, a corresponding level(s) of the herniation(s) as indicated by the anatomy indicators and/or condition indicators, such that the output illustration provides a visual representation to improve the understanding of the reported findings for a layperson.

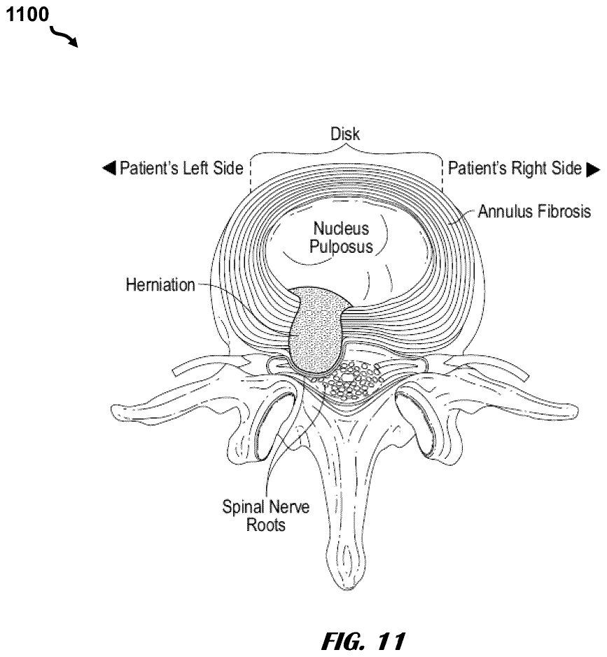

FIG. 11 depicts the graphical user interface 1100 displaying a machine-generated illustration for an axial view (top-down view) of the spine at level L4-L5. The illustration includes additional image elements of portions of the anatomy at the portion of the spine, medical conditions, and annotations indicating some of the areas of interest as described in the input report (e.g., input radiology report). The generative AI may generate the illustration image according to anatomy indicators and/or condition indicators, where the generated illustration contains elements that depict or indicate a location of anatomy (e.g., left and right sides of body for orientation, nucleus pulposus, annulus fibrosis, disk) or conditions and a severity of the condition (e.g., herniation). The generative AI generates the elements of the illustration of the graphical user interface 1100 based upon, for example, a corresponding level(s) of the herniation(s) as indicated by the anatomy indicators and/or condition indicators, such that the output illustration provides a visual representation to improve the understanding of the reported findings for the layperson.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, attributes, or memory contents. Information, arguments, attributes, data, etc. may be passed, forwarded, or transmitted via any suitable means including memory sharing, message passing, token passing, network transmission, etc.

The actual software code or specialized control hardware used to implement these systems and methods is not limiting of the invention. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-Ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of non-limiting description and understanding and are not intended to be limiting, with the true scope and spirit being indicated by the following claims.

What is claimed is:

1. A computer-implemented method for enhancing radiology reports having medical imaging data, comprising:
obtaining, by a computer, magnetic resonance imaging (MRI) imagery data containing one or more MRI images generated from an MRI imaging device;
identifying, by the computer in an illustration database containing a plurality of medical illustration image files, a set of one or more medical illustration images based upon a set of one or more MRI slices of the one or more MRI images, the set of one or more medical illustration images being identified according to text of an input source report having the MRI imagery data, wherein for each medical illustration image the computer identifies a medical illustration image based upon at least one of an anatomical descriptor or a condition indicator in the input source report and associated with the medical illustration image;
for each MRI image, generating, by the computer, an output image by combining an MRI image with the medical illustration image as identified in the illustration database using the one or more MRI slices of the MRI image; and
generating, by the computer, an output report having one or more output images.

2. The method according to claim 1, further comprising generating, by the computer, a report summary based upon the text of the input source report, wherein the computer generates the output report having the one or more output images and the report summary.

3. The method according to claim 1, wherein selecting the one or more MRI slices includes, for each MRI slice of the one or more MRI slices, generating, by the computer, a colorized instance of the MRI slice.

4. The method according to claim 3, wherein generating the colorized instance includes:
identifying, by the computer, in the MRI slice a portion of an anatomy having a medical condition according to the condition indicator of the input source report; and
updating, by the computer, a coloring of the portion of the anatomy having the medical condition.

5. The method according to claim 3, wherein generating the colorized instance includes receiving, by the computer, a user input indicating a coloring a portion of an anatomy in the MRI slice having a medical condition.

6. The method according to claim 1, wherein generating the output image includes updating, by the computer, the MRI imagery data containing the one or more MRI images based upon merging the MRI image and an overlay of the medical illustration image.

7. The method according to claim 6, further comprising generating, by the computer, a refined image according to a graph representation of one or more image features, including an anatomical accuracy value and an illustration clarity value for the medical illustration image.

8. The method according to claim 1, wherein identifying the set of one or more medical illustration images includes selecting, by the computer, from the illustration database at least one of an axial image or a sagittal image based upon herniation data of the condition indicator.

9. The method according to claim 1, further comprising:
receiving, by the computer, the input source report having the MRI imagery data in a healthcare message data structure having a standard format; and
extracting, by the computer, the input source report and the MRI imagery data from the healthcare message data structure.

10. The method according to claim 1, further comprising selecting, by the computer, the one or more MRI slices from the MRI imagery data according to one or more input from a user device, each MRI slice includes an overlay corresponding to one or more condition indicators of the input source report.

11. A system for enhancing radiology reports having medical imaging data, the system comprising:
an illustration database configured to store a plurality of medical illustration image files corresponding to a plurality of portions of human anatomy; and
a computer comprising at least one processor configured to:
obtain magnetic resonance imaging (MRI) imagery data containing one or more MRI images generated from an MRI imaging device;
identify in the illustration database a set of one or more medical illustration images based upon a set of one or more MRI slices of the one or more MRI images, the set of one or more medical illustration images being identified according to text of an input source report having the MRI imagery data, wherein for each medical illustration image the computer identifies a medical illustration image based upon at least one of an anatomical descriptor or a condition indicator in the input source report and associated with the medical illustration image;
for each MRI image, generate an output image by combining an MRI image with the medical illustration image as identified in the illustration database using the one or more MRI slices of the MRI image; and
generate an output report having one or more output images.

12. The system according to claim 11, wherein the computer is further configured to generate a report summary based upon the text of the input source report, and wherein the computer generates the output report having the one or more output images and the report summary.

13. The system according to claim 11, wherein, when selecting the one or more MRI slices, the computer is further configured to, for each MRI slice of the one or more MRI slices, generate a colorized instance of the MRI slice.

14. The system according to claim 13, wherein, when generating the colorized instance, the computer is further configured to:
identify in the MRI slice a portion of an anatomy having a medical condition according to the condition indicator of the input source report; and
update a coloring of the portion of the anatomy having the medical condition.

15. The system according to claim 13, wherein, when generating the colorized instance, the computer is further configured to receive a user input indicating a coloring a portion of an anatomy in the MRI slice having a medical condition.

16. The system according to claim 11, wherein, when generating the output image, the computer is further configured to update the MRI imagery data containing the one or more MRI images based upon merging the MRI image and an overlay of the medical illustration image.

17. The system according to claim 16, wherein the computer is further configured to generate a refined image according to a graph representation of one or more image features, including an anatomical accuracy value and an illustration clarity value for the medical illustration image.

18. The system according to claim 11, wherein, when identifying the set of one or more medical illustration images, the computer is further configured to select from the illustration database at least one of an axial image or a sagittal image based upon herniation data of the condition indicator.

19. The system according to claim 11, wherein the computer is further configured to:
receive the input source report having the MRI imagery data in a healthcare message data structure having a standard format; and
extract the input source report and the MRI imagery data from the healthcare message data structure.

20. The system according to claim 11, wherein the computer is further configured to select the one or more MRI slices from the MRI imagery data according to one or more input from a user device, each MRI slice includes an overlay corresponding to one or more condition indicators of the input source report.
